(19) United States
(12) Patent Application Publication
(10) Pub. No.: US 2010/0016449 A1
(43) Pub. Date: Jan. 21, 2010

(54) Formulations with Improved Bioavailability

(75) Inventors: George W. Gereg, JR., Bethel, CT (US); Xiaohui Mei, Highland Mills, NY (US); Li Zhong, New Milford, CT (US)

Correspondence Address: MICHAEL P. MORRIS;BOEHRINGER INGELHEIM USA CORPORATION, 900 RIDGEBURY ROAD, P O BOX 368, RIDGEFIELD, CT, 06877-0368, US

(73) Assignee: BOEHRINGER INGELHEIM INTERNATIONAL GMBH, Ingelheim am Rhein (DE)

(21) Appl. No.: 12/518,286

(22) PCT Filed: Dec. 7, 2007
(86) PCT No.: PCT/US07/86709

§ 371 Date: Sep. 22, 2009

Related U.S. Application Data
(60) Provisional application No. 60/871,251, filed on Dec. 21, 2006.

(51) Int. Cl. A61K 47/08

(52) U.S. Cl. 514/772

(57) ABSTRACT

Disclosed are solid oral formulations with improved dissolution and bioavailability for poorly water soluble pharmaceutical compounds.

### BACKGROUND OF THE INVENTION

1. Technical Field

This invention relates to solid oral formulations with improved bioavailability for poorly water soluble pharmaceutical compounds.

2. Background Information

Many active ingredients for pharmaceuticals possess less than desirable solubility and bioavailability pharmacokinetic properties. Their pharmaceutical dosage forms are therefore often difficult to design.

Aryl- and heteroaryl-substituted ureas have been described as inhibitors of cytokine production and effective therapeutics in cytokine-mediated diseases including inflammatory and autoimmune diseases. Examples of such compounds are reported in U.S. Pat. Nos. 6,080,763 and 6,319,921, and WO 00/55139.

US 2004/265378 and WO 03/082262 disclose compositions prepared by melt granulation using a single binder. Vitamin E TPGS (d-alpha-tocopheryl polyethylene glycol 1000 succinate), is a non-ionic pharmaceutically acceptable surfactant. However, Vitamin E TPGS, being a waxy material, is too sticky to be processed for typical solid dosage form due to its low melting point (37-41° C.).

Formulations of particular heteroaryl urea compounds have also been discovered to be plagued with less than desirable adherency characteristics. These formulations have been found to suffer from the tendency of materials to stick to compression dies and/or punch faces, as well as to stick to powder conduits, filling tubes, and other processing chambers.

There is a need therefore for pharmaceutical formulations with improved solubility and diminished adherency characteristics, which provide better oral bioavailability of the drug as well as allow for efficient preparation of dosage forms.

### BRIEF SUMMARY OF THE INVENTION

The present invention discloses formulations of poorly water soluble pharmaceutical compounds, and processes for manufacturing such formulations, that provide for improved solubility and/or bioavailability. In particular, advantageous oral dosage formulations of these compounds are provided.

It is therefore an object of the invention to provide pharmaceutical formulations that improve dissolution and oral absorption of poorly water soluble compounds, which comprise: (a) providing a pharmaceutically active ingredient; (b) providing a non-ionic surfactant with a lower melting point; (c) providing a polymer with a higher melting point; (d) optionally providing other pharmaceutically acceptable excipients; (e) melt granulating using melted mixture of (b) and (c) as the binder.

### DESCRIPTION OF THE FIGURES

FIG. 1: FIG. 1 shows in vitro dissolution profiles of compound 1 besilate form formulations in 900 mL pH 4.0 citric-phosphate buffer (USP paddle method at 50 rpm, 37° C.): Example #1 (); Example #2 (◯); Example #3 (▾); conventional tablet (Δ).

### DETAILED DESCRIPTION OF THE INVENTION

The process of the invention comprises:

combining the pharmaceutically active ingredient, optionally with other excipients, with the binders, the binders comprising at least one non-ionic surfactant with a lower melting point and one polymer with a higher melting point; granulating at a temperature of about 40˜100° C.

The invention provides for melt granulation for incorporation of a non-ionic surfactant such as cetyl alcohol, polyoxyl castor oil, polyoxyl 2 cetyl ether, polyoxyl 10 cetyl ether, polyoxyl 20 cetyl ether, polyoxy 23 lauryl ether, polyoxyl 2 stearyl ether, polyoxyl 10 stearyl ether, polyoxyl 20 stearyl ether, lauroyl macrogolglycerides, stearoyl macrogolglycerides, preferably Vitamin E TPGS (d-alpha-tocopheryl polyethylene glycol 1000 succinate), into solid dosage forms. The non-ionic surfactant has been shown to be very helpful in increasing solubility/dissolution of particular pharmaceutically active ingredients listed herein below. However non-ionic surfactants such as Vitamin E TPGS, are waxy materials and are too sticky to be processed for typical solid dosage form due to their low melting point (37-41° C.). The current invention utilizes the process of melt granulation to incorporate non-ionic surfactants into the solid oral formulation. More specifically, the invention combines a non-ionic surfactant with a secondary binder. Preferred secondary binders include polyethylene glycol (MW>3000), poloxamer, anionic emulsifying wax, carnauba wax, glyceryl behenate, glyceryl palmitostearate, hydrogenated castor oil, microcrystalline wax, non-ionic emulsifying wax, stearyl alcohol, white beeswax and yellow beeswax. Most preferred is PEG 6000 which is a polymer with relatively higher melting point (55-60° C.). The resulting granules will not be sticky and can be easily processed.

Other excipients can be added. Nonlimiting examples include: pH modifiers such as tartaric acid, fumaric acid, citric acid, lactic acid, malic acid, glutarmic acid, monobasic sodium phosphate, ascorbic acid, diethanolamine, monoethanolamine, potassium bicarbonate, potassium citrate, potassium hydroxide, sodium bicarbonate, sodium citrate dehydrate, sodium hydroxide, triethanolamine, tromethamine; disintegrants such as alginic acid, calcium phosphate, carboxymethylcellulose calcium, carboxymethylcellulose sodium, cellulose, chitosan, colloidal silicon dioxide, croscarmellose sodium, crospovidone, docusate sodium, guar gum, hydroxypropyl cellulose, magnesium aluminum silicate, methylcellulose, microcrystalline cellulose, povidone, sodium alginate, sodium starch glycolate, starch; glidants such as calcium phosphate, calcium silicate, cellulose, colloidal silicon dioxide, magnesium silicate, magnesium trisilicate, silicon dioxide, starch, talc; lubricants such as calcium stearate, glycerin monostearate, glyceryl behenate, glyceryl palmitostearate, hydrogenated castor oil, hydrogenated vegetable oil, light mineral oil, magnesium lauryl sulfate, magnesium stearate, medium-chain triglycerides, mineral oil, poloxamer, polyethylene glycol, sodium benzoate, sodium chloride, sodium lauryl sulfate, sodium stearyl fumarate, stearic acid, talc, zinc stearate.

The order of addition can vary and will be apparent to those of ordinary skill in the art. As a non-limiting example, the drug can be granulated alone or together with other excipients; the binders can be melted first or mixed with the drug and/or other excipients prior to granulation. Thus, the binders can be melted first then the drug added, then granulation or, the drug and binders added first, then the heat is raised 40-100 during granulation causing only the binders to melt.

The pharmaceutically active ingredient within the scope of the present invention are any poorly soluble compounds. Preferred are those chosen from, but not limited to, those disclosed in U.S. Pat. Nos. 6,319,921, 6,358,945, 5,716,972, 5,686,455, 5,656,644, 5,593,992, 5,593,991, 5,663,334, 5,670,527, 5,559,137, 5,658,903, 5,739,143, 5,756,499, 6,277,989, 6,340,685, and 5,716,955; and PCT applications WO 92/12154, WO 94/19350, WO 95/09853, WO 95/09851, WO 95/09847, WO 95/09852, WO 97/25048, WO 97/25047, WO 97/33883, WO 97/35856, WO 97/35855, WO 97/36587, WO 97/47618, WO 97/16442, WO 97/16441, WO 97/12876, WO 98/25619, WO 98/06715, WO 98/07425, WO 98/28292, WO 98/56377, WO 98/07966, WO 98/56377, WO 98/22109, WO 98/24782, WO 98/24780, WO 98/22457, WO 98/52558, WO 98/52559, WO 98/52941, WO 98/52937, WO 98/52940, WO 98/56788, WO 98/27098, WO 98/47892, WO 98/47899, WO 98/50356, WO 98/32733, WO 99/58523, WO 99/01452, WO 99/01131, WO 99/01130, WO 99/01136, WO 99/17776, WO 99/32121, WO 99/58502, WO 99/58523, WO 99/57101, WO 99/61426, WO 99/59960, WO 99/59959, WO 99/00357, WO 99/03837, WO 99/01441, WO 99/01449, WO 99/03484, WO 99/15164, WO 99/32110, WO 99/32111, WO 99/32463, WO 99/64400, WO 99/43680, WO 99/17204, WO 99/25717, WO 99/50238, WO 99/61437, WO 99/61440, WO 00/26209, WO 00/18738, WO 00/17175, WO 00/20402, WO 00/01688, WO 00/07980, WO 00/07991, WO 00/06563, WO 00/12074, WO 00/12497, WO 00/31072, WO 00/31063, WO 00/23072, WO 00/31065, WO 00/35911, WO 00/39116, WO 00/43384, WO 00/41698, WO 00/69848, WO 00/26209, WO 00/63204, WO 00/07985, WO 00/59904, WO 00/71535, WO 00/10563, WO 00/25791, WO 00/55152, WO 00/55139, WO 00/17204, WO 00/36096, WO 00/55120, WO 00/55153, WO 00/56738, WO 01/21591, WO 01/29041, WO 01/29042, WO 01/62731, WO 01/05744, WO 01/05745, WO 01/05746, WO 01/05749, WO 01/05751, WO 01/27315, WO 01/42189, WO 01/00208, WO 01/42241, WO 01/34605, WO 01/47897, WO 01/64676, WO 01/37837, WO 01/38312, WO 01/38313, WO 01/36403, WO 01/38314, WO 01/47921, WO 01/27089, DE 19842833, and JP 2000 86657 whose disclosures are all incorporated herein by reference in their entirety.

Of particular interest are those pharmaceutically active ingredients disclosed in U.S. Pat. Nos. 6,319,921, 6,358,945, 6,277,989, 6,340,685, WO 00/12074, WO 00/12497, WO 00/59904, WO 00/71535, WO 01/64676, WO 99/61426, WO 00/10563, WO 00/25791, WO 01/37837, WO 01/38312, WO 01/38313, WO 01/38314, WO 01/47921, WO 99/61437, WO 99/61440, WO 00/17175, WO 00/17204, WO 00/36096, WO 98/27098, WO 99/00357, WO 99/58502, WO 99/64400, WO 99/01131, WO 00/43384, WO 00/55152, WO 00/55139 and WO 01/36403.

Preferred, are those pharmaceutically active ingredients disclosed in U.S. Pat. Nos. 6,319,921 and 6,358,945.

More preferred, the pharmaceutically active ingredient is chosen from:

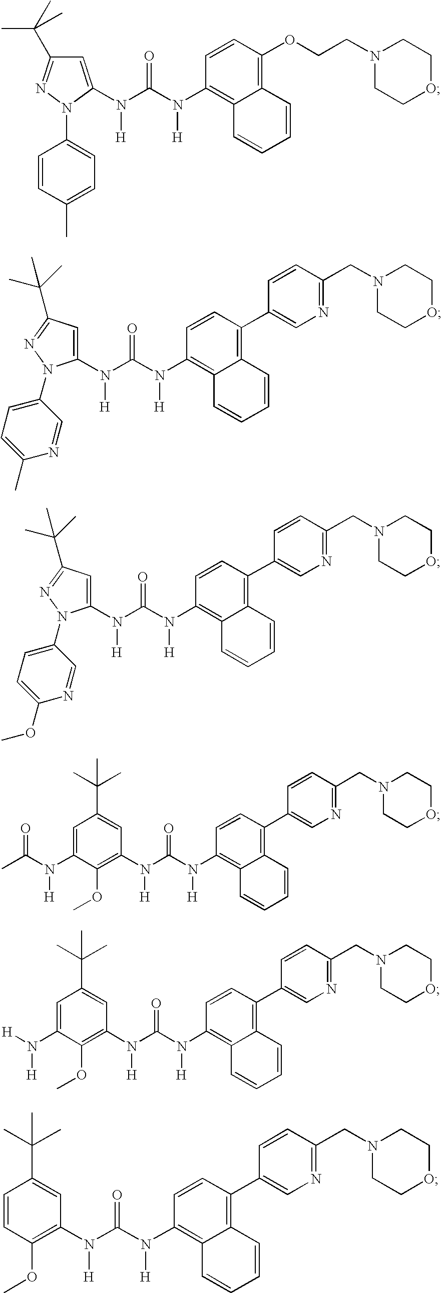

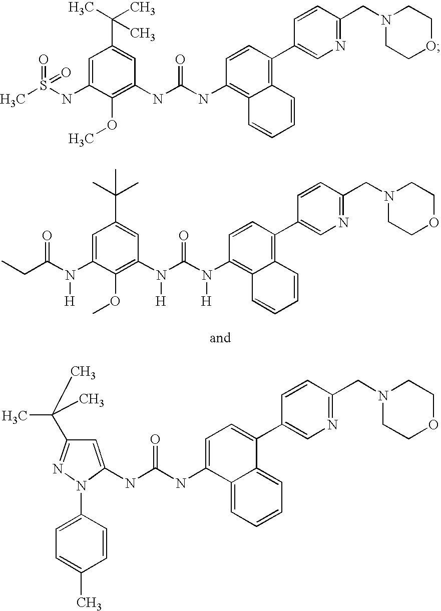

or the pharmaceutically acceptable salts thereof.

Final dosage form: the formulation can be either filled into hard shell capsules or compressed into tablets.

In order that this invention be more fully understood, the following examples are set forth. These examples are for the purpose of illustrating preferred embodiments of this invention, and are not to be construed as limiting the scope of the invention in any way.

### Examples

Following is a typical procedure for the current method:

1. Blend drug and fillers (e.g. Microcrystalline Cellulose) together.

2. Melt and mix low melting point binder (e.g. d-alpha-tocopheryl polyethylene glycol 1000 succinate) and high melting point binder (e.g. Polyethylene Glycol 6000) at a suitable temperature (e.g. 70° C.).

3. Add the pre-blend powder to the melted binders and granulate at a suitable temperature (e.g. 60° C.) in a high shear granulator.

4. Cool the granules down to room temperature and equilibrate for at least 12 hours.

5. Mill the granules and further mix with glidant (e.g. colloidal silicon dioxide) and lubricant (e.g. magnesium stearate).

6. Fill the final blend into hard gelatin capsules.

### Example 1

| Ingredient | mg/capsule | % (w/w) |
|---|---|---|
| Compound 1 besilate form | 127.5 | 26.3 |
| Microcrystalline Cellulose | 179 | 36.9 |
| d-alpha-tocopheryl polyethylene | 50 | 10.3 |
| glycol 1000 succinate | | |
| Polyethylene glycol 6000 | 100 | 20.6 |
| Tartaric acid | 25 | 5.2 |
| Colloidal Silicon Dioxide | 3.5 | 0.7 |
| Total | 485 | 100.0 |

### Example 2

| Ingredient | mg/capsule | % (w/w) |
|---|---|---|
| Compound 1 besilate form | 127.5 | 26.3 |
| Microcrystalline Cellulose | 179 | 36.9 |
| Lauroyl Macrogolglycerides | 50 | 10.3 |
| Polyethylene glycol 6000 | 100 | 20.6 |
| Tartaric acid | 25 | 5.2 |
| Colloidal Silicon Dioxide | 3.5 | 0.7 |
| Total | 485 | 100.0 |

### Example 3

| Ingredient | mg/capsule | % (w/w) |
|---|---|---|
| Compound 1 besilate form | 127.5 | 26.3 |
| Microcrystalline Cellulose | 179 | 36.9 |
| Stearoyl Macrogolglycerides | 50 | 10.3 |
| Polyethylene glycol 6000 | 100 | 20.6 |
| Tartaric acid | 25 | 5.2 |
| Colloidal Silicon Dioxide | 3.5 | 0.7 |
| Total | 485 | 100.0 |

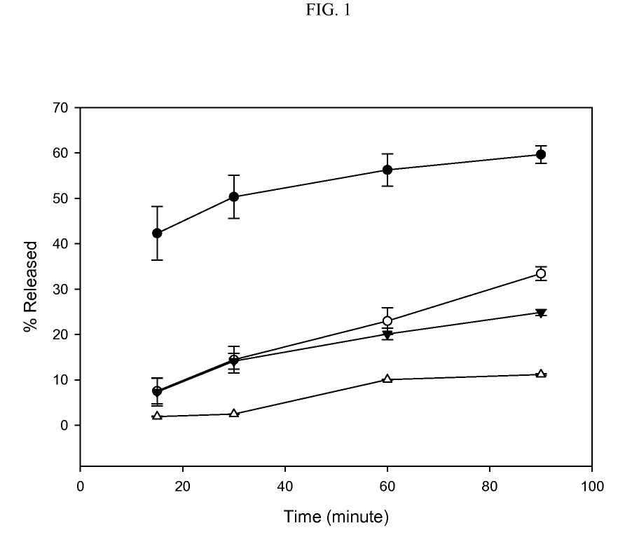

The formulations (examples #1-3) showed significantly improved in vitro dissolution compared to a conventional tablet (FIG. 1). The cross-over PK study in dog (n=6) demonstrated that formulation example #1 provided higher bioavailability compared to a reference solution formulation (Table 1).

TABLE 1
Summary of pharmacokinetic parameters after oral dosing of formulation
Example #1 and a reference solution formulation (dose = 200 mg).

| | | |
|---|---|---|
| Formulation | Solution ^{b} (n = 6) | Example #1 (n = 6) |

| | | | | |
|---|---|---|---|---|
| Cₘₐₓ ^{a} (ng/mL) | 3,992 | (44) | 5,231 | (70) |
| tₘₐₓ ^{a} (h) | 2 | (1.5-2) | 2 | (0.67-3) |
| AUC₀₋₄₈ ^{a} | 22,348 | (49) | 38,189 | (81) |
| (ng · h/mL) | | | | |

^{a} Cₘₐₓ and AUC₀₋₄₈ are presented as mean (% RSD) and tₘₐₓ is presented as median (range).
^{b} 40 mg/mL of Compound 1 in a mixture of 80% PEG 400/20% Ethanol. (w/w)

Compound 1: 1-[5-tert-Butyl-2-p-tolyl-2H-pyrazol-3-yl]-3{4-[6-(morpholin-4-ylmethyl)pyridine-3-yl]naphthalen-1-yl}urea.

All literature and patent documents cited in this application, including US provisional application 60/663,324 are all incorporated herein by reference in their entirety.

1. A pharmaceutical formulation comprising: a pharmaceutically active ingredient; optionally pharmaceutically acceptable excipients; and a melted mixture of an non-ionic surfactant with a lower melting point and a polymer with a higher melting point as a binder, wherein the lower and higher melting points are with respect to the non-ionic surfactant and polymer.

2. The pharmaceutical formulation according to claim 1 wherein the non-ionic surfactant is chosen from cetyl alcohol, polyoxyl castor oil, polyoxyl 2 cetyl ether, polyoxyl 10 cetyl ether, polyoxyl 20 cetyl ether, polyoxy 23 lauryl ether, polyoxyl 2 stearyl ether, polyoxyl 10 stearyl ether, polyoxyl 20 stearyl ether, lauroyl macrogolglycerides, stearoyl macrogolglycerides and Vitamin E TPGS; the polymer is chosen from polyethylene glycol (MW>3000), poloxamer, anionic emulsifying wax, carnauba wax, glyceryl behenate, glyceryl palmitostearate, hydrogenated castor oil, microcrystalline wax, non-ionic emulsifying wax, stearyl alcohol, white beeswax and yellow beeswax.

3. The pharmaceutical formulation according to claim 2 wherein the non-ionic surfactant is Vitamin E TPGS and the polymer is PEG 6000.

4. A process of making a pharmaceutical formulation comprising:
combining a pharmaceutically active ingredient, optionally with other excipients, with a binders, the binders comprising at least one non-ionic surfactant with a lower melting point and one polymer with a higher melting point;
granulating at a temperature of about 40˜100° C.;
wherein the binders can be melted first or mixed with the active ingredient and/or other excipients prior to granulation.

5. The process according to claim 4 wherein the non-ionic surfactant is chosen from cetyl alcohol, polyoxyl castor oil, polyoxyl 2 cetyl ether, polyoxyl 10 cetyl ether, polyoxyl 20 cetyl ether, polyoxy 23 lauryl ether, polyoxyl 2 stearyl ether, polyoxyl 10 stearyl ether, polyoxyl 20 stearyl ether, lauroyl macrogolglycerides, stearoyl macrogolglycerides and Vitamin E TPGS; the polymer is chosen from polyethylene glycol (MW>3000), poloxamer, anionic emulsifying wax, carnauba wax, glyceryl behenate, glyceryl palmitostearate, hydrogenated castor oil, microcrystalline wax, non-ionic emulsifying wax, stearyl alcohol, white beeswax and yellow beeswax.

6. The pharmaceutical formulation according to claim 5 wherein the non-ionic surfactant is Vitamin E TPGS and the polymer is PEG 6000.
